(19) United States
(12) Patent Application Publication
(10) Pub. No.: US 2016/0348054 A1
(43) Pub. Date: Dec. 1, 2016

(54) Biogas stirring device for anaerobic tank

(71) Applicant: Bioland Environmental Technologies Group Corp. Ltd. (Beijing Branch), Beijing (CN)

(72) Inventors: Junying Shi, Beijing (CN); Yichao Ping, Beijing (CN); Ying Wang, Beijing (CN)

(73) Assignee: Bioland Environmental Technologies Group Corp. Ltd. (Beijing Branch)

(21) Appl. No.: 15/212,251

(22) Filed: Jul. 17, 2016

(30) Foreign Application Priority Data
Jul. 22, 2015 (CN) 201520534866.0

(51) Int. Cl. C12M 1/04; A01C 3/02; B01F 15/02

(52) U.S. Cl. CPC C12M 27/04; B01F 15/026; A01C 3/026; B01F 2215/0073; F16K 31/06

(57) ABSTRACT

A biogas stirring device for an anaerobic tank includes: a biogas compressor, a biogas distributer, and gas inlet valves; wherein the biogas distributer includes a main pipe and a plurality of sub-pipes communicating with the main pipe; wherein a tank wall of the anaerobic tank has a plurality of installing holes corresponding to an amount of the sub-pipes; each of the installing holes is provided with one of the gas inlet valves, and an inlet end of each of the gas inlet valves communicates with one of the sub-pipes; an outlet end of each of the gas inlet valves communicates with an inside area of the anaerobic tank. Advantages of the present invention are as follows: the present invention effectively solves blockage of the conventional exhaust pipes; the present invention is simple in structure, low in cost, convenient in installation and easy in maintenance.

### CROSS REFERENCE OF RELATED APPLICATION

The present invention claims priority under 35 U.S.C. 119(a-d) to CN 201520534866.0, filed Jul. 22, 2015.

### BACKGROUND OF THE PRESENT INVENTION

Field of Invention

The present invention relates to a stirring device, and more particularly to a biogas stirring device for an anaerobic tank.

Description of Related Arts

Anaerobic fermentation refers to stabilizing the waste under anaerobic conditions by microbial metabolic activity, accompanied by methane and CO₂ produced.

Stirring is an indispensable operation to promote anaerobic fermentation, wherein efficient stirring can increase the chance of contact between materials and microorganism; evenly distribute temperature and materials within the system; prevent local acid accumulation; rapidly discharge biological generated gases such as hydrogen sulfide and methane which inhibit anaerobic activities; fully break generated dross, etc.

Stirring devices of conventional field of anaerobic fermentation include the following three ways:

(I) hydraulic Stirring

Hydraulic stirring generally uses flowing fluid generated by a pump for stirring, whose device comprises: a draft tube screw pump inside or outside an anaerobic digestion tank or a centrifugal pump outside the anaerobic digestion tank, a screw pump, and pipelines. In addition, hydraulic jet may be arranged inside the anaerobic digestion tank for enhancing stirring effect. Problems of the hydraulic stirring are: the stirring intensity is small, so the hydraulic stirring is only suitable for material with a lower concentration.

(II) Mechanical Stirring

Mechanical stirring uses mechanisms with various installation forms for mixing the materials through various forms of mechanical movements. Problems of the mechanical stirring are: blind angle exists during stirring, and stirring is incomplete.

(III) Gas Stirring

Gas stirring is achieved through bubble movements among the materials. In biogas fermentation, stirring mainly relies on biogas circulating. Conventionally, a gas stirring device comprises: a biogas compressor outside an anaerobic digestion tank, and staggered exhaust pipes inside the anaerobic digestion tank. Operations of the gas stirring device are: after compressed by the biogas compressor, the biogas enters each of the exhaust pipes, and then enters the anaerobic digestion tank, in such a manner the materials in the tank are pushed by the biogas for stirring and cycling. Problems of the gas stirring device are: because of the staggered exhaust pipes, liquid in the tank may reversely flows into the pipe opening, resulting in blockage of the exhaust pipes.

### SUMMARY OF THE PRESENT INVENTION

An object of the present invention is to provide a biogas stirring device for an anaerobic tank for overcoming the above problems

Accordingly, in order to accomplish the above object, the present invention provides a biogas stirring device for an anaerobic tank, comprising: a biogas compressor (1), a biogas distributer (2), and gas inlet valves (3);

wherein the biogas distributer (2) and the biogas compressor (1) are placed outside the anaerobic tank;

wherein the biogas distributer (2) comprises a main pipe (2-1) and a plurality of sub-pipes (2-2) communicating with the main pipe (2-1); an inlet end of the main pipe (2-1) communicates with an outlet end of the biogas compressor (1) through a pipeline (4); a tank wall of the anaerobic tank has a plurality of installing holes corresponding to an amount of the sub-pipes (2-2); each of the installing holes is provided with one of the gas inlet valves (3), and an inlet end of each of the gas inlet valves (3) communicates with one of the sub-pipes (2-2); an outlet end of each of the gas inlet valves (3) communicates with an inside area of the anaerobic tank.

Preferably, the tank wall, where the installing holes are provided, is a bottom tank wall.

Preferably, the gas inlet valves (3) are electromagnetic valves.

Advantages of the present invention are as follows.

(1) The present invention effectively solves blockage of the conventional exhaust pipes; meanwhile, due to the gas stirring, a stirring intensity is large, stirring is sufficient, and materials may have a large range of concentrations.

(2) The present invention is simple in structure, low in cost, convenient in installation and easy in maintenance, and is suitable for wide application.

These and other objectives, features, and advantages of the present invention will become apparent from the following detailed description, the accompanying drawings, and the appended claims.

### BRIEF DESCRIPTION OF THE DRAWINGS

FIG. 1 is a sketch view of a biogas stirring device for an anaerobic tank according to the present invention.

FIG. 2 is a sketch view of a biogas compressor according to the present invention.

FIG. 3 is a sketch view of a biogas distributer according to the present invention.

FIG. 4 is a sketch view of gas inlet valves according to the present invention.

### DETAILED DESCRIPTION OF THE PREFERRED EMBODIMENT

Referring to the drawings, the present invention is further illustrated.

Referring to FIG. 1, a biogas stirring device for an anaerobic tank is provided, comprising: a biogas compressor 1, a biogas distributer 2, and gas inlet valves 3. The three elements are illustrated in detail as follows.

(1) the Biogas Compressor 1

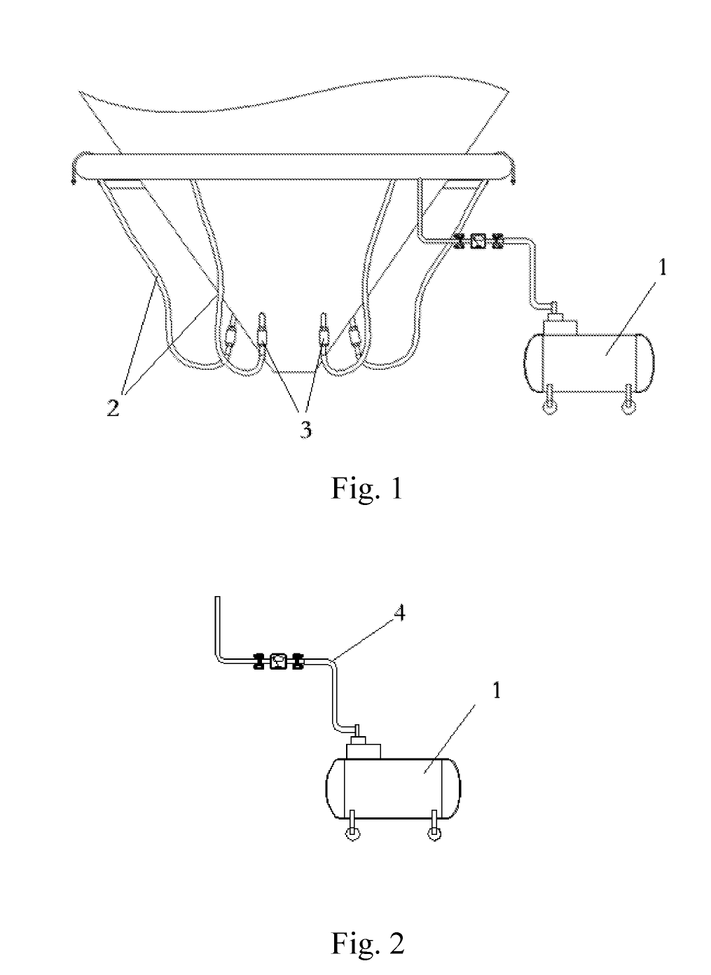

Referring FIG. 2, a sketch view of the biogas compressor 1 according to the present invention is shown, wherein the biogas compressor 1 is placed outside the anaerobic tank, mainly for supplying the biogas distributer 2 with compressed biogas, which is a power source of the biogas stirring device.

(2) The Biogas Distributer 2

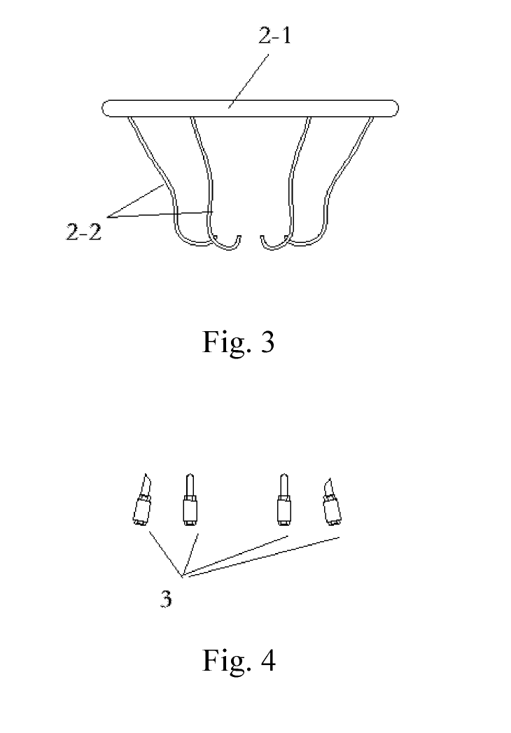

Referring to FIG. 3, a sketch view of the biogas distributer 2 according to the present invention is shown, wherein the biogas distributer 2 is also placed outside the anaerobic tank. Referring to FIG. 3, the biogas distributer 2 comprises a main pipe 2-1 and a plurality of sub-pipes 2-2 communicating with the main pipe 2-1; wherein an inlet end of the main pipe 2-1 communicates with an outlet end of the biogas compressor 1 through a pipeline 4; and an outlet end of each of the sub-pipes 2-2 communicates with a corresponding one of the gas inlet valves 3.

(3) the Gas Inlet Valves 3

Referring to FIG. 4, a sketch view of the gas inlet valves 3 according to the present invention is shown, wherein a tank wall of the anaerobic tank has a plurality of installing holes corresponding to an amount of the sub-pipes 2-2; and each of the installing holes is provided with one of the gas inlet valves 3. As an example, in FIG. 3, the biogas distributer 4 has four sub-pipes 2-2. As a result, there are four gas inlet valves 3 in FIG. 4. An inlet end of each of the gas inlet valves 3 communicates with one of the sub-pipes 2-2; and an outlet end of each of the gas inlet valves 3 communicates with an inside area of the anaerobic tank. In practice, the gas inlet valves 3 are electromagnetic valves. Furthermore, for enhancing stirring intensity and effect, the tank wall, where the installing holes are provided, is a bottom tank wall, in such a manner that each of the gas inlet valves 3 upwardly sprays a gas flow into the anaerobic tank, for gas stirring inside the anaerobic tank.

Principles of the above biogas stirring device are as follows.

(1) For running the whole device, the gas inlet valves 3 on the tank wall are closed and the biogas compressor 1 is started, in such a manner that gas pressure in pipelines between the biogas compressor 1 and the gas inlet valves 3 reaches a working pressure. Then the gas inlet valves 3 on the tank wall are opened, wherein the gas outputted from the biogas compressor 1 firstly passes through the biogas distributer 2, and finally enters the anaerobic tank through the gas inlet valves 3, so as to stir materials inside the anaerobic tank.

In addition, the biogas distributer 2 is placed outside the anaerobic tank and the gas inlet valves 3 are provided on the tank wall, so there is no pipe inside the anaerobic tank. Therefore, liquid inside the anaerobic will never block the pipes, which avoids pipeline blockage.

(2) For stopping the stirring device, the gas inlet valves 3 on the tank wall are firstly closed for avoiding that the liquid inside the anaerobic passes through the gas inlet valves 3 and blocks the sub-pipes 2-2 of the biogas distributer 2. Then the biogas compressor 1 is stopped for stopping the stirring device. As a result, pipeline blockage is avoided, and operation of the stirring device is kept normal.

Therefore, advantages of the present invention are as follows.

(1) The present invention effectively solves blockage of the conventional exhaust pipes; meanwhile, due to the gas stirring, a stirring intensity is large, stirring is sufficient, and materials may have a large range of concentrations.

(2) The present invention is simple in structure, low in cost, convenient in installation and easy in maintenance, and is suitable for wide application.

One skilled in the art will understand that the embodiment of the present invention as shown in the drawings and described above is exemplary only and not intended to be limiting.

It will thus be seen that the objects of the present invention have been fully and effectively accomplished. Its embodiments have been shown and described for the purposes of illustrating the functional and structural principles of the present invention and is subject to change without departure from such principles. Therefore, this invention includes all modifications encompassed within the spirit and scope of the following claims.

What is claimed is:

1. A biogas stirring device for an anaerobic tank, comprising: a biogas compressor (1), a biogas distributer (2), and gas inlet valves (3);
wherein the biogas distributer (2) and the biogas compressor (1) are placed outside the anaerobic tank;
wherein the biogas distributer (2) comprises a main pipe (2-1) and a plurality of sub-pipes (2-2) communicating with the main pipe (2-1); wherein an inlet end of the main pipe (2-1) communicates with an outlet end of the biogas compressor (1) through a pipeline (4); a tank wall of the anaerobic tank has a plurality of installing holes corresponding to an amount of the sub-pipes (2-2); each of the installing holes is provided with one of the gas inlet valves (3), and an inlet end of each of the gas inlet valves (3) communicates with one of the sub-pipes (2-2); an outlet end of each of the gas inlet valves (3) communicates with an inside area of the anaerobic tank.

2. The biogas stirring device, as recited in claim 1, wherein the tank wall, where the installing holes are provided, is a bottom tank wall.

3. The biogas stirring device, as recited in claim 1, wherein the gas inlet valves (3) are electromagnetic valves.
